(19) United States
(12) Patent Application Publication
(10) Pub. No.: US 2026/0240181 A1
(43) Pub. Date: Aug. 20, 2026

(54) SPRAY DRIED MILK OR MILK-BASED POWDER COMPOSITION AND METHODS THEREOF

(71) Applicant: Kerry Group Services International Ltd, Co. Kerry (IE)

(72) Inventor: Vojislav Vojinovic, Hoersholm (DK)

(21) Appl. No.: 19/159,514

(22) PCT Filed: Feb. 27, 2024
(86) PCT No.: PCT/EP2024/054917

§ 371 Date: Aug. 25, 2025

(30) Foreign Application Priority Data
Feb. 28, 2023 (EP) 23159104.1
Jul. 3, 2023 (EP) 23183039.9

(51) Int. Cl. A23C 9/12; A23C 1/04; A23C 9/18; C12N 9/24

(52) U.S. Cl. CPC A23C 9/1206; A23C 1/04; A23C 9/18; C12N 9/2402; C12Y 302/01023

(57) ABSTRACT

The present disclosure generally relates to a spray dried milk or milk-based powder composition comprising galacto-oligosaccharides (GOS) and low levels of lactose, and a method of making said composition.

### TECHNICAL FIELD

The present disclosure generally relates to a spray dried milk or milk-based powder composition comprising a beta-galactosidase enzyme, galacto-oligosaccharides (GOS), low levels of lactose, maltodextrin and/or sodium chloride free, and a method of making said composition. Additionally, the present disclosure also relates to use of the beta-galactosidase enzyme for improving spray drying and/or for improving mass yield of a spray dried composition herein disclosed and/or reduce browning of spray dried milk or milk-based powder composition, preferably wherein the term improving or reducing is in relation to the use of a beta-galactosidase enzyme without transgalactosylating activity or in relation to the non-use of a beta-galactosidase regardless of the transgalactosylating activity.

### BACKGROUND

Reduction of lactose in milk is commonly achieved by lactase treatment in which lactose is hydrolyzed to glucose and galactose. However, it is known to be difficult to convert a mix of milk or milk-based substrate, glucose, and galactose into a powder, such as a spray dried powder. A common solution to this problem is to add maltodextrin or similar ingredients. A disadvantage of adding these aids is the increase of carbohydrates in the powder milk, which is seen as undesired from a nutritional perspective.

WO2010149557 discloses addition of lactase to milk either shortly before spray drying or in the spray drier as a separate stream, such that no interaction of the enzyme with the milk composition takes places prior to the drying step, therefore preventing any unwanted hydrolysis of lactose to take place. The hydrolysis of lactose occurs in reconstituted milk.

WO2016071500 discloses a spray dried composition comprising an enzyme which is a beta-galactosidase having transgalactosylating activity and spray drying aids, such as maltodextrin and/or sodium chloride (NaCl).

WO2021180702 discloses a method for producing a lactose reduced dairy powder comprising treating a lactose comprising milk-based substrate with a combination of two enzymes, a lactase to reduce lactose to low levels and a beta-galactosidase enzyme having transgalactosylating activity to form GOS in a powder.

In conclusion, low lactose milk or free lactose milk is difficult to spray dry and as a result, losses occur, meaning that the yield obtained is less than the desired. The impact in the industry is significant. Therefore, there is a need for a spray dried milk or milk-based composition, wherein the composition has reduced concentration of lactose and improved yields.

### SUMMARY

The present document describes a different approach to what is known, in which a spray dried composition comprising a GOS-forming lactase also named as beta-galactosidase having transgalactosylating activity, is used to convert lactose into GOS prior to the spray drying step. The spray drying of milk or milk-based substrate with a beta-galactosidase having transgalactosylating activity instead of a beta-galactosidase not having this activity (or significantly not having this activity) avoids losses in mass yield, while simultaneously increases the proportion of oligosaccharides or galacto-oligosaccharides (GOS). The present approach contrasts with the disclosure of WO2010149557, in which the beta-galactosidase enzyme is not a beta-galactosidase having transgalactosylating activity and wherein no interaction of the enzyme with the milk composition occurs prior to drying.

Additionally, the present approach leads to a reduction in the levels needed for spray drying aids, i.e., maltodextrin or sodium chloride, or even their complete avoidance, as the enzyme increases the proportion of oligosaccharides or galacto-oligosaccharides (GOS) and reduces the proportion of glucose and galactose. This contrasts with the disclosure of WO2016071500, in which the purpose of WO2016071500 was to have a spray dried product comprising a lactase and maltodextrin and/or sodium chloride, wherein said spray dried aids allow the enzyme to be physically stable and retain their transgalactosylating activity. However, in the present disclosure the enzyme produces GOS prior to the spray drying step; therefore, the physical stability and retention of activity after the spray drying step is not a concern of this disclosure. In fact, the present disclosure avoids the maintenance of the physical stability and retention of activity of the enzyme after the spraying step thereby preventing a possible degradation of GOS by the enzyme, which would lead to a composition with less, if any, intestinal health benefits.

Finally, this disclosure or invention is made possible due to the reduction of lactose and production of GOS by a single beta-galactosidase enzyme having transgalactosylating activity (a GOS-forming lactase) prior to the spray drying step, which contrasts with the disclosure made in WO2021180702, wherein two different enzymes are used.

### Spray Dried Milk Powder Composition or Spray Dried Milk-Based Powder Composition

The first aspect of the present disclosure or invention relates to a spray dried milk powder composition or spray dried milk-based powder composition comprising:
a beta-galactosidase enzyme;
galacto-oligosaccharides, preferably at least 1% w_{galacto-oligosaccharides}/w_{composition};
less than 5% w_{lactose}/w_{composition};
free, or substantially free, of maltodextrin;
free, or substantially free, of sodium chloride.

In the context of the present disclosure or invention, the composition herein disclosed is free, or substantially free, of maltodextrin. To be free, or substantially free, of maltodextrin means that no maltodextrin, or other spray drying aids, is added to the milk or milk-based substrate prior to spray drying. As a result, maltodextrin is absent from the composition obtained after the spray drying step. Therefore, to be “free, or substantially free, of maltodextrin” means that “maltodextrin is absent from the composition”.

In the context of the present disclosure or invention, the composition herein disclosed is free, or substantially free, of sodium chloride. To be free, or substantially free, of sodium chloride means that no sodium chloride, or other spray drying aids, is added to the milk or milk-based substrate prior to spray drying. As a result, sodium chloride is absent from the composition obtained after the spray drying step. Therefore, to be “free, or substantially free, of sodium chloride” means that “sodium chloride is absent from the composition”.

The first aspect may include several embodiments as below described. These embodiments may be combined.

In a preferred embodiment, the beta-galactosidase enzyme may have transgalactosylating activity.

In a preferred embodiment, the beta-galactosidase enzyme may be a Bifidobacterium beta-galactosidase, preferably the beta-galactosidase enzyme may be a Bifidobacterium bifidum beta-galactosidase, more preferably the beta-galactosidase enzyme may have at least 90%, at least 95%, at least 96%, at least 97%, at least 98%, at least 99% or 100% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 90% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 95% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 96% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 97% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 98% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 99% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 100% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the composition herein disclosed may comprise at least 8% w/w of galacto-oligosaccharides, or at least 9% w/w, or at least 10% w/w, or least 11% w/w, or least 12% w/w, wherein w/w is w_{galacto-oligosaccharides}/w_{composition}. Preferably, said composition 8%-30% w/w of galacto-oligosaccharides or 10%-25% w/w or 11%-20% w/w or 12%-15% w/w, wherein w/w is w_{galacto-oligosaccharides}/w_{composition}.

In a preferred embodiment, the composition herein disclosed may comprise 4.5% w/w or less of lactose, or 4% w/w or less of lactose, such as 0.1% w/w-4.5% w/w or 0.2% w/w-4.0% w/w or 0.3% w/w-3.5% w/w or 0.4% w/w-3% w/w or 0.5% w/w-2.5% w/w, or 0.6% w/w-2.0% w/w or 0.7% w/w-1.5% w/w or 0.8% w/w-1.0% w/w, wherein w/w is w_{lactose}/w_{composition}.

In a preferred embodiment, the composition wherein disclose may have a water content of below 6% w/w, wherein w/w is w_{water} content/w_{composition}.

In a preferred embodiment, the composition may be a spray dried skimmed milk powder composition or spray dried skimmed milk-based powder composition.

In a preferred embodiment, the composition may comprise at most 1.5% of fat or at most 1% of fat, such as 1.5%-0.1% or 1.0%-0.1% or 1.5%-0.4% or 1.0-0.4% or 1.5%-0.8% or 1.0%-0.8% or 0.8%-0.4% of fat, wherein % is % w_{fat}/w_{milk or milk-based substrate}.

In a preferred embodiment, the composition may comprise protein sources, carbohydrate sources, coloring, minerals, vitamins, probiotics, prebiotics, supplements, among others.

### Method of Producing or Making the Spray Dried Milk Powder Composition or Spray Dried Milk-Based Powder Composition Herein Disclosed

A second aspect of the present disclosure or invention relates to a method for producing a spray-dried milk powder composition or spray dried milk-based powder composition, as above disclosed, wherein the method comprises the following steps:
providing a milk or milk-based substrate comprising at least 1% w_{lactose}/w_{milk or milk-based substrate} and at most 1.5% w_{fat}/w_{milk or milk-based substrate};
mixing the milk or milk-based substrate with a beta-galactosidase enzyme;
allowing the beta-galactosidase enzyme to generate galacto-oligosaccharides, preferably at least 1% w_{galacto-oligosaccharides}/w_{composition};
spray drying the mix comprising milk or milk-based substrate, beta-galactosidase enzyme and galacto-oligosaccharides;
obtaining a spray dried milk powder composition or milk-based substrate powder composition as disclosed in the first aspect.

The second aspect may include several embodiments as below described. These embodiments may be combined.

In a preferred embodiment, the beta-galactosidase enzyme used in the method May have transgalactosylating activity.

In a preferred embodiment, the beta-galactosidase enzyme used in the method may be a Bifidobacterium beta-galactosidase, preferably the beta-galactosidase enzyme may be a Bifidobacterium bifidum beta-galactosidase, more preferably the beta-galactosidase enzyme may have at least 90%, at least 95%, at least 96%, at least 97%, at least 98%, at least 99% or 100% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 90% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 95% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 96% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 97% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 98% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 99% sequence identity to SEQ ID NOs 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme used in the method may have transgalactosylating activity and at least 100% sequence identity to SEQ ID NOS 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the step of allowing the beta-galactosidase enzyme to generate galacto-oligosaccharides may be carried out such that at least 8% w/w of galacto-oligosaccharides are generated, or at least 9% w/w, or at least 10% w/w, or least 11% w/w, or least 12% w/w, wherein w/w is w_{galacto-oligosaccharides}/w_{composition}. Preferably, said composition 8%-30% w/w of galacto-oligosaccharides are generated or 10%-25% w/w or 11%-20% w/w or 12%-15% w/w, wherein w/w is w_{galacto-oligosaccharides}/w_{composition}.

In a preferred embodiment, the method may be carried out such that the composition obtained from the method may comprise less than 5% w/w or less of lactose or 4.5% w/w or less of lactose, or 4% w/w or less of lactose, such as 0.1% w/w-4.5% w/w or 0.2% w/w-4.0% w/w or 0.3% w/w-3.5% w/w or 0.4% w/w-3% w/w or 0.5% w/w-2.5% w/w, or 0.6% w/w-2.0% w/w or 0.7% w/w-1.5% w/w or 0.8% w/w-1.0% w/w, wherein w/w is w_{lactose}/w_{composition}.

In a preferred embodiment, the step of mixing the milk or milk-based substrate with a beta-galactosidase enzyme may be carried out with at least 0.1 g/L or at most 10 g/L or with less than 10 g/L, preferably with 0.1-10 g/L or 0.1-9 g/L or 0.1-8 g/L, more preferably with 0.2-7 g/L or 0.3-8 g/L, even more preferably with 0.3-5 g/L or 0.3-3 g/L or 0.5-1 g/L, wherein g/L is g_{beta-galactosidase of enzyme}/Lₘᵢₗₖ or g_{beta-galactosidase of enzyme}/L_{milk-substrate}. Alternatively, in the context of the present disclosure, 1 g (including the tested dosages range of 0.1 g to 10 g) of enzyme dosage per liter of milk or per liter of milk-based substrate may refer or refers to 1 g of a commercial enzyme product, such as Saphera® Fiber or Nurica, having a defined activity (as per certificate of analysis by the supplier) in 3000-3300 LAU-C/g or 600-650 U/g applied to 1 L of milk, respectively. Additionally, “g/L_{milk-based substrate} of beta-galactosidase enzyme” and “g_{beta-galactosidase enzyme}/L_{milk based substrate}” have the meaning and are, in the entire disclosure interchangeable. In a preferred embodiment, the method is carried out such that the composition may have a water content of below 6% w/w, such as 4%/w, wherein w/w is w_{water content}/w_{composition}.

In a preferred embodiment, the step of providing a milk or milk-based substrate may be carried out with a skimmed milk or a skimmed milk-based substrate, preferably wherein the milk or milk-based substrate comprises 1.5%-0.1% or 1.0%-0.1% or 1.5%-0.4% or 1.0%-0.4% or 1.0%-0.8% of fat, more preferably 0.8%-0.4% of fat. The milk used had a fat content of 1.5% or less such as 1.5%-0.4% of fat, preferably 1.0% or less such as 1.0%-0.4% of fat. A fat content as the one herein disclosed was found to be the preferred fat content to avoid the addition of spray drying aids, such as maltodextrin or sodium chloride.

In a preferred embodiment, the method may be carried out milk selected from mammal milk, such as cow's milk, sheep's milk, goat's milk, buffalo's milk or camel's milk.

In a preferred embodiment, the method herein disclosed may be carried out in the absence of a spray drying aid, such as maltodextrin or sodium chloride; or in the absence of an added spray drying aid, such as added maltodextrin or added sodium chloride. Therefore, no maltodextrin and/or sodium chloride is added.

In a preferred embodiment, the step of mixing the milk or milk-based substrate with a beta-galactosidase enzyme and the step of allowing the beta-galactosidase enzyme to generate galacto-oligosaccharides may be carried out at the same time.

In a preferred embodiment, the step of allowing the beta-galactosidase enzyme to generate galacto-oligosaccharides may be carried out between 1 h-24 h, preferably 8 h-20 h at 2° C.-10° C.; or 1 h-3 h at 50° C.-60° C.

In a preferred embodiment, the method herein disclosed is method for producing a spray-dried milk powder composition or spray dried milk-based powder composition, wherein the method comprises the following steps:
a) providing an aqueous composition comprising less than 30%, such as 1-25% or 4-20% or 10-20% by weight of non-fat milk solids including lactose;
b) adding a beta-galactosidase to the composition, wherein the beta-galactosidase is any one of the enzymes herein disclosed;
c) allowing the beta-galactosidase to react for 10-240 minutes at 50-65° C.;
d) treating the composition after step c) at a temperature of below 70° C., such as at 40° C. or 68° C. for 10-150 seconds; or treating the composition after step c) at a temperature 70-150° C. for 10-150 seconds; treating the composition after step c) at a temperature 4-10° C. for 12-48 h;
e) spray drying the composition after step d),
wherein:
optionally, the beta-galactosidase is not inhibited by changing the pH to outside the optimum activity of the enzyme;
optionally, no maltodextrin is present or if maltodextrin is added then the weight ratio of maltodextrin and sorbate in the composition is not 28.6:71.4;
optionally, no desalted whey powder or lactose powder is used with raw milk to obtain a feed liquid;
optionally a sweetener can be present or added to one of the steps of this process;
or no sweetener is present or added if desalted whey powder or lactose powder is used with raw milk or milk or milk-based substrate;
the beta-galactosidase enzyme having transgalactosylating activity and at least 90% sequence identity to SEQ ID NOs 1, 2, 3, 4, 5, 6, 7, 8, 9, 10, 11, 12, 13, 14, 15, 16, 17 or 18.

### Use of a Beta-Galactosidase Enzyme Having Transgalactosylating Activity

A third aspect of the present disclosure or invention relates to the use of a beta-galactosidase enzyme having transgalactosylating activity for improving spray drying of milk or milk-based substrate and/or for improving mass yield of a spray dried milk powder composition or spray dried milk-based powder composition and/or for reducing browning of spray dried milk or milk-based powder composition.

In the context of the present aspect, but also in the context to the present disclosure or invention, the term “improving” or “reducing” is in relation to the use of a beta-galactosidase enzyme without transgalactosylating activity or in relation to the non-use of a beta-galactosidase regardless of the transgalactosylating activity.

In a preferred embodiment, the beta-galactosidase enzyme may be a Bifidobacterium beta-galactosidase; preferably the beta-galactosidase enzyme may be a Bifidobacterium bifidum beta-galactosidase, more preferably the beta-galactosidase enzyme may have at least 90%, at least 95%, at least 96%, at least 97%, at least 98%, at least 99% or 100% sequence identity to SEQ ID Nos 1, 2, 3, 4, 5, 6, 7, 8, 9, 10, 11, 12, 13, 14, 15, 16, 17, or 18, even more preferably wherein the sequence is SEQ ID NO 1 or SEQ ID NO 7 or SEQ ID NO 7 glycosylated.

In a preferred embodiment, the beta-galactosidase enzyme may have transgalactosylating activity and at least 90% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 95% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 96% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, th beta-galactosidase enzyme may have transgalactosylating activity and at least 97% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 98% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 99% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, the beta-galactosidase enzyme may have transgalactosylating activity and at least 100% sequence identity to SEQ ID Nos 1, 2, 3, 4, 5, 6, 7, 8, 9, 10, 11, 12, 13, 14, 15, 16, 17, or 18; preferably the sequence may be SEQ ID NO 1 or SEQ ID NO 7 or SEQ ID NO 7 glycosylated.

In a preferred embodiment, at least 0.1 g/L or at most 10 g/L or with less than 10 g/L of the beta-galactosidase enzyme may be used, preferably with 0.1-10 g/L or 0.1-9 g/L or 0.1-8 g/L, more preferably with 0.2-7 g/L or 0.3-8 g/L, even more preferably with 0.3-5 g/L or 0.3-3 g/L or 0.5-1 g/L, wherein g/L is g_{beta-galactosidase of enzyme}/Lₘᵢₗₖ or g_{beta-galactosidase of enzyme}/L_{milk-substrate}. Alternatively, in the context of the present disclosure, 1 g (including the tested dosages range of 0.1 g to 10 g) of enzyme dosage per liter of milk or per liter of milk-based substrate may refer or refers to 1 g of a commercial enzyme product, such as Saphera® Fiber or Nurica, having a defined activity (as per certificate of analysis by the supplier) in 3000-3300 LAU-C/g or 600-650 U/g applied to 1 L of milk, respectively. Additionally, “g/L_{milk-based substrate} of beta-galactosidase enzyme” and “g_{beta-galactosidase enzyme}/L_{milk based substrate}” have the meaning and are, in the entire disclosure interchangeable. In an embodiment, 2 g/L to 6 g/L or 3-4 g/L of beta-galactosidase enzyme may be used, wherein g/L is g_{beta-galactosidase of enzyme}/Lₘᵢₗₖ or g_{beta-galactosidase of enzyme}/L_{milk-substrate}.

### Definitions

In the context of the present disclosure, the following definitions and concepts apply.

“Galacto-oligosaccharides (GOS)” are non-digestible carbohydrates, in particular oligosaccharides composed of 2 or more monosaccharide units, which are produced by the enzymatic treatment of lactose, by a beta-galactosidase. Lactose is not considered or defined as GOS. This enzymatic reaction produces a mixture of oligosaccharides of varying lengths linked by glycosidic bonds, unreacted lactose, and monomeric sugars (D-glucose and D-galactose).

GOS has the beneficial effect of acting as prebiotics by enhancing the growth and selectively stimulating the proliferation and activity of beneficial colonic microorganisms, such as bacteria from the genus Bifidobacterium, to give physiological benefits to the consumer, either at a short-, medium- or long-term. The genus Bifidobacterium is also associated to health-promoting effects, for example, ability to repopulate the intestinal flora in individuals who have had their intestinal flora disturbed by for example intake of antibiotics and/or ability to outcompete potential harmful intestinal micro-organisms.

The established health effects have contributed to the growing and continued interest in GOS as food ingredients for various types of food.

GOS generally comprise a chain of galactose units that arise through consecutive trans-galactosylation reactions, with a terminal glucose unit or a terminal fructose unit if sucrose is present in the reaction mixture. The DP of GOS can vary quite markedly, depending mainly on the type of the beta-galactosidase used and the conversion degree of lactose.

A “beta-galactosidase” (EC 3.2.1.23) enzyme performs two different reactions, hydrolysis of lactose and transgalactosylation. In the first step, the glycosidic linkage between glucose and galactose in the lactose substrate is broken, and glucose is released and galactosyl enzyme intermediate is formed. In the second step, this new covalent bond in the galactosyl enzyme intermediate will be then attached by a nucleophile, either water or sugar. If the incoming nucleophile is water, then the resulting reaction is called hydrolysis and lead to the release of galactose. If the incoming nucleophile is another sugar, such as lactose, the resulting reaction is called galactosyl transfer reaction which leads to galacto-oligosaccharide formation (GOS formation). GOS formation by beta-galactosidase enzymes may be favored, for example, by high concentrations of lactose or low water activity.

“Spray drying” is a method of converting a liquid or a slurry into a dry powder by rapidly drying the liquid or slurry with a hot gas, which may be, for example, air.

“Milk” is to be understood as the lacteal secretion obtained by milking of any mammal, such as cows, sheep, goats, buffaloes or camels. In a preferred embodiment, the milk is cow's milk. The term milk also includes protein/fat solutions made of plant materials, e.g., soy milk, provided lactose is present.

“Milk substrate” or “milk-based substrate” may be any raw and/or processed milk material that can be subjected to fermentation according to the method of the invention. Thus, useful milk substrates include, but are not limited to, solutions/-suspensions of any milk or milk like products comprising lactose, preferably comprising at least 0.002% (0.002 g/100 ml) of lactose, such as whole or low-fat milk, skim milk, buttermilk, reconstituted milk powder, condensed milk, dried milk, whey, whey permeate, whey protein concentrate, acid whey, cream, fermented milk products, such as yogurt or cheese. Preferably, milk, milk substrate or milk-based substrate is skimmed milk, skimmed milk substrate or skimmed milk-based substrate. The “milk substrate” or “milk-based substrate” may originate from any mammal, e.g., being substantially pure mammalian milk, or reconstituted milk powder. Typically, the term “milk substrate” or “milk-based substrate” refers to a raw or processed milk material that is processed further to produce a dairy product. Prior to fermentation, the “milk substrate” may be homogenized and pasteurized according to methods known in the art.

“Powder milk” is a dried version of milk with typically 4-6% moisture content.

“Spray dried milk or milk-based powder composition” means spray dried milk powder composition or spray dried milk-based powder composition.

“Sequence identity” for amino acids as used herein refers to the sequence identity calculated as (n_{ref}−n_{dif}). 100/n_{ref}, wherein n_{dif} is the total number of non-identical residues in the two sequences when aligned and wherein n_{ref} is the number of residues in one of the sequences. In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DETAILED DESCRIPTION

The present document generally relates to a spray dried milk or milk-based powder composition comprising a beta-galactosidase enzyme, galacto-oligosaccharides (GOS) formed in-situ, low levels of lactose, maltodextrin and/or sodium chloride free, and a method of making said composition. Additionally, the present disclosure also relates to use of the beta-galactosidase enzyme for improving spray drying and/or for improving mass yield of a spray dried composition herein disclosed, preferably wherein the term “improving” or “reducing” is in relation to the use of a beta-galactosidase enzyme without transgalactosylating activity or in relation to the non-use of a beta-galactosidase regardless of the transgalactosylating activity.

In the context of the present invention, any Bifidobacterium derived beta-galactosidase or Bifidobacterium beta-galactosidase may be used, such a Bifidobacterium bifidum derived beta-galactosidase or Bifidobacterium bifidum beta-galactosidase. Several documents disclose examples of Bifidobacterium derived beta-galactosidase and/or Bifidobacterium bifidum derived beta-galactosidase.

For example, the sequences disclosed in WO2013182686 or WO2020117548 can be herein used, as well as methods to obtain and/or treat said sequences, such as after expressing said sequences. In particular, SEQ ID Nos 1 to 6 of WO2013182686 correspond to SEQ ID Nos 1 to 6, respectively of the present disclosure or invention. In particular, SEQ ID Nos 1 to 5 of WO2020117548 correspond to SEQ ID Nos 1 to 5, respectively, of the present disclosure or invention. Preferably, SEQ ID No 1 of WO2013182686 or SEQ ID No 1 of WO2020117548 is used or SEQ ID No 1 of the present disclosure or invention is used. Alternatively, Dupont™ Danisco® Nurica™ may be used. In the context of the present disclosure or invention, SEQ ID No 1 and Nurica™ are used or may be used interchangeably.

In alternative or in addition, the sequences of WO2018210820 can also be herein used, as well as methods to obtain and/or treat said sequences, such as after expressing said sequences and/or submitting said sequences to a glycosylation step as described in Example of WO2018210820. In particular, SEQ ID Nos 1 to 12 of WO2018210820 correspond to SEQ ID Nos 7 to 18, respectively, of the present disclosure or invention. Preferably SEQ ID No 1 of WO2018210820 corresponds to SEQ ID No 7 of the present invention, wherein the sequence has been submitted to a glycosylation or glycation step (glycosylation and glycation are used interchangeably) under the conditions explained in WO2018210820. Alternatively, Saphera® Fiber available from Novozymes A/S may be used. In the context of the present invention, SEQ ID No: 7 glycosylated and Saphera® Fiber are used or may be used interchangeably.

In the present disclosure, the method for quantification of monosaccharides, dissacharides and GOS may be carried out as follows:
GOS, as well as mono- and disaccharides were analyzed by high-performance anion-exchange chromatography with pulsed amperometric detection (HPAE-PAD), on a Dionex ICS-5000, ICS-6000 or Integrion system (Thermo Fischer Scientific, Waltham, MA, USA). The systems were all equipped with a Dionex™ CarboPac™ SA10 column (4 mm×250 mm, 6 μM), and a EGC KOH Eluent Generator Cartridge, that was used to generate a gradient starting with 1 mM KOH for 10 min, which was linear increased to 20 mM over 20 min, and to 100 mM over further 20 min, where it was held for 20 min, before reverting to 1 mM in 0.1 min, keeping this for 20 min. Flow rate was 1.0 ml/min and column temperature 30° C. Water (MQ) was purified from a Milli-Q water purifier (Millipore, Burlington, MA, USA), to 18.2 MΩ×cm and a TOC<5 ppb. An AXP pump was used for feeding 0.5 ml/min 300 mM NaOH post column to boost detector signal. Fructose, sucrose, galactose, glucose, lactose, trehalose (Sigma-Aldrich Chemie Gmbh, Munich, Germany), allolactose, 1,3-β-D-Galactosyl-D-glucose, 6′-Galactosyllactose, 4′-Galactosyllactose, and 3′-Galactosyllactose (Carbosynth Limited, Compton, UK), were analyzed for external standard quantification, based on peak areas. Bimuno® Daily (Clasado Limited, Shinfield, Reading, UK) with GOS were diluted in MQ used as quality control samples.
Samples and control (1.0 g) were quenched with 2 ml 96% ethanol and frozen. After thawing they were diluted with 2 ml MQ and a subsample centrifuged at 21 000×g for 10 min, the supernatant was then further diluted to 500 times with MQ containing 0.02% (w/v) sodium azide (Sigma-Aldrich), and 3 μl sub-samples injected in the HPAE-PAD system.

There are, however, alternative methods for quantifying monosaccharides, disaccharides and GOS, which have been, for example, disclosed in WO2013/182686, WO2015/132402, WO2020/117548 or WO2018/210820. The AOAC 2001.02 method is also an alternative. Regardless of the method used, what is relevant is that within the same example or within comparative examples, the quantification is made in the same way.

### EXAMPLES

### Example 1—Preparation of Spray Dried Milk Powders

Three samples of milk, in particular skimmed milk, with a dry matter content of 8.75% w/w, were treated with a lactase as shown in Table 1 and further incubated for 24 h at 6° C.

TABLE 1

| | Sample 1 | Sample 2 | Control |
|---|---|---|---|
| Dosage of beta-galactosidase | 3.0 | 4.0 | — |
| enzyme having transgalactosylating | | | |
| activity, in g/L | | | |
| Dosage of control lactase, in g/L | — | — | 0.5 |

In example 1, the beta-galactosidase enzyme having transgalactosylating activity (or GOS-forming lactase) used was Saphera® Fiber from Novozymes A/S. Alternatively, any other GOS-forming lactase as the ones herein disclosed may be used as well, such as a sequence having transgalactosylating activity and at least 90% sequence identity to SEQ ID NOs 1, 2, 3, 4, 5, 6, 7, 8, 9, 10, 11, 12, 13, 14, 15, 16, 17 or 18. Identical or similar results are expected.

In this example the control lactase used was NOLA® Fit 5500 from Chr. Hansen A/S. Alternatively, other control lactase enzymes may be used such as Maxilact LGi5000 from DSM or Ha-La® from Chr. Hansen A/S, both beta-galactosidases from Kluyveromyces lactis. Identical or similar results are expected.

After incubation, samples 1, 2 and the control were spray dried on pilot spray dryer, in particular a SPX AnHydro PSD-52 with an internal diameter of 1 m and 60 degree cone, to residual water content of approx. 4% and a water activity of approx. 19%±1%. No maltodextrin or other spray drying aids were added. The parameters used to spray dry the samples were: inlet temperature: 180° C. outlet temperature: 80° C., milk feed-flow: 4.8 kg/h, drying gas rate: 175 kg/h, Dryer pressure: −5 mBarg/−50 mmWC.

The spray dried samples were individually recovered below the cyclone, weighed, and analyzed as shown in Table 2.

TABLE 2

| | Sample 1 | Sample 2 | Control |
|---|---|---|---|
| Initial dry matter content in total, in g | 875 | 875 | 875 |
| Final dry matter content in total, in g | 594 | 624 | 540 |
| Spray dried skimmed milk powder with | 618.7 | 650.0 | 562.6 |
| approx. 4% (w/w) water content, in g | | | |
| Mass Yield, in % | 67.9 | 71.3 | 61.7 |

Table 2 shows that the hydrolysis of lactose with a beta-galactosidase enzyme having transgalactosylating activity (or GOS-forming lactase) leads to a higher recovery of a spray dried milk powder composition from the spray drying process. In particular, it was possible to recover between 10%-16% more spray dried milk powder composition when a GOS-forming lactase was used versus a control lactase. Therefore, a higher mass yield was obtained for samples 1 and 2 versus the control. Furthermore, it was also possible to recover around 10% more spray dried milk powder composition when a dosage of 4 g/L of GOS-forming lactase was used versus 3 g/L.

### Example 2—Evaluation of GOS and Lactose Content

Samples 1, 2 and the control were further studied to determine their respective concentration of GOS and lactose—Table 3. The samples were stored at 25° C. until they were dry, after which GOS and lactose quantifications were carried out on the samples and control, as described above. In the context of the present disclosure, a sample is dry if the water content is below 6% w/w, which is the stability criterium for milk powders.

TABLE 3

| | Sample 1 | Sample 2 | Control |
|---|---|---|---|
| GOS, in mg/g | 109.8 | 119.1 | 8.6 |
| GOS, in % w/w | ~11 | ~12 | ~0.9 |
| Lactose, in mg/g | 42.5 | 27.3 | 19.9 |
| Lactose, in % w/w | ~4 | ~3 | ~2 |

### Example 3—Evaluation of Browning

Upon obtaining the spray dried powders, samples and the control were presented to 28 panelists. Prior to be this, samples and control were anonymized and randomized. Panelists were asked to identify which was darker (or whiter) by observing them under controlled white light. The panelists consistently identified samples 1 and 2 to be whiter than the control. Therefore, the spray dried samples (1 and 2) are also less prone to browning than the control.

SEQUENCE LISTING

SEQ ID NO: 1
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKEDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKT
SEQ ID NO: 2
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGAL
SEQ ID NO: 3
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQ
SEQ ID NO: 4
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKEDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQI
SEQ ID NO: 5
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSDRVKPYTYDFAPVGATFVK
VTVTNADTTTPSGVVCAGLTEIELKTAT
SEQ ID NO: 6
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSDRVKPYTYDFAPVGATFVK
VTVTNADTTTPSGVVCAGLTEIELKTATSKFVTNTSAALSSLTVNGTKVSDSVLAAGSYNTPAIIADVKAEGEGNASVTVLPAHDNVIRVI
TESEDHVTRKTFTINLGTEQEF
SEQ ID NO: 7
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGNVTMNLTTKVANDTKAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGADKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVD
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSERVKPYTYDFAPVGATFVK
VTVTNADTTTPSGVVCAGLTEIELKTATSKFVTNTSAALSSLTVNGTKVSDSVLAAGSYNTPAIIADVKAEGEGNASVTVLPAHDNVIRVI
TESEDHVTRKTFTINLGTEQEFPADSDERD
SEQ ID NO: 8
MKKPLGKIVASTALLISVAFSSSIASAAVEDATRSDSTTQMSSTPEVAYSSAVDSKQNRTSDFDANWKFMLSDSVQAQDPAFDDSAWQQVD
LPHDYSITQKYSQSNEAESAYLPGGTGWYRKSFTIDRDLAGKRIAINFDGVYMNATVWFNGVKLGTHPYGYSPFSFDLTGNAKFGGENTIV
VKVENRLPSSRWYSGSGIYRDVTLTVTDGVHVGNNGVAIKTPSLATQNGGDVTMNLTTKVANDTEAAANITLKQTVFPKGGKTDAAIGTVT
TASKSIAAGASADVTSTITAASPKLWSIKNPNLYTVRTEVLNGGKVLDTYDTEYGFRWTGFDATSGFSLNGEKVKLKGVSMHHDQGSLGAV
ANRRAIERQVEILQKMGVNSIRTTHNPAAKALIDVCNEKGVLVVEEVFDMWNRSKNGNTEDYGKWFGQAIAGDNAVLGGDKDETWAKFDLT
STINRDRNAPSVIMWSLGNEMMEGISGSVSGFPATSAKLVAWTKAADSTRPMTYGDNKIKANWNESNTMGDNLTANGGVVGTNYSDGANYD
KIRTTHPSWAIYGSETASAINSRGIYNRTTGGAQSSDKQLTSYDNSAVGWGAVASSAWYDVVQRDFVAGTYVWTGFDYLGEPTPWNGTGSG
AVGSWPSPKNSYFGIVDTAGFPKDTYYFYQSQWNDDVHTLHILPAWNENVVAKGSGNNVPVVVYTDAAKVKLYFTPKGSTEKRLIGEKSFT
KKTTAAGYTYQVYEGSDKDSTAHKNMYLTWNVPWAEGTISAEAYDENNRLIPEGSTEGNASVTTTGKAAKLKADADRKTITADGKDLSYIE
VDVTDANGHIVPDAANRVTFDVKGAGKLVGVDNGSSPDHDSYQADNRKAFSGKVLAIVQSTKEAGEITVTAKADGLQSSTVKIATTAVPGT
STEKTVRSFYYSRNYYVKTGNKPILPSDVEVRYSDGTSDRQNVTWDAVSDDQIAKAGSFSVAGTVAGQKISVRVTMIDEIGALLNYSASTP
VGTPAVLPGSRPAVLPDGTVTSANFAVHWTKPADTVYNTAGTVKVPGTATVFGKEFKVTATIRVQRSQVTIGSSVSGNALRLTQNIPADKQ
SDTLDAIKDGSTTVDANTGGGANPSAWTNWAYSKAGHNTAEITFEYATEQQLGQIVMYFFRDSNAVRFPDAGKTKIQISADGKNWTDLAAT
ETIAAQESSDRVKPYTYDFAPVGATFVKVTVTNADTTTPSGVVCAGLTEIELKTATSKFVTNTSAALSSLTVNGTKVSDSVLAAGSYNTPA
IIADVKAEGEGNASVTVLPAHDNVIRVITESEDHVTRKTFTINLGTEQEFPADSDERDYPAADMTVTVGSEQTSGTATEGPKKFAVDGNTS
TYWHSNWTPTTVNDLWIAFELQKPTKLDALRYLPRPAGSKNGSVTEYKVQVSDDGTNWTDAGSGTWTTDYGWKLAEFNQPVTTKHVRLKAV
HTYADSGNDKFMSASEIRLRKAVDTTDISGATVTVPAKLTVDRVDADHPATFATKDVTVTLGDATLRYGVDYLLDYAGNTAVGKATVTVRG
IDKYSGTVAKTFTIELKNAPAPEPTLTSVSVKTKPSKLTYVVGDAFDPAGLVLQLNYDDDSTGTVTWNTQTAGDFTFKPALDAKLKVTDKT
VTVTYQGKSAVIDITVSQPAPTVSKTDLDKAIKAIEAKNPDSSKYTADSWKTFADAMAHAKAVIADDSATQQDVDNALKALTDAYAGLTEK
TPEPAPVSKSELDKKIKAIEAEKLDGSKYTAESWKAFETALAHAKAVIASDSATQQNVDAALGALTSARDGLTEKGEVKPDPKPEPGTVDK
AALDKAVKKVEAEKLDGSKYTADSWKAFETALAHAKAVIGNANSTQFDIDNALSMLNDARAALKEKPGRIIAIIDGSALSKTGASVAIIAS
VAAAMLAVGAGVMALRRKRS
SEQ ID NO: 9
MKKPLGKIVASTALLISVAFSSSIASAIEDATRSDSTTQMSSTPEVAYSSAVDSKQNRTSDFDANWKFMLSDSVQAQDPAFDDSAWQQVDL
PHDYSITQKYSQSNEAESAYLPGGTGWYRKSFTIDRDLAGKRIAINFDGVYMNATVWFNGVKLGTHPYGYSPFSFDLTGNAKFGGENTIVV
KVENRLPSSRWYSGSGIYRDVTLTVTDGVHVGNNGVAIKTPSLATQNGGDVTMNLTTKVANDTEAAANITLKQTVFPKGGKTDAAIGTVTT
ASKSIAAGASADVTSTITAASPKLWSIKNPNLYTVRTEVLNGGKVLDTYDTEYGFRWTGFDATSGFSLNGEKVKLKGVSMHHDQGSLGAVA
NRRAIERQVEILQKMGVNSIRTTHNPAAKALIDVCNEKGVLVVEEVFDMWNRSKNGNTEDYGKWFGQAIAGDNAVLGGDKDETWAKFDLTS
TINRDRNAPSVIMWSLGNEMMEGISGSVSGFSATSAKLVAWTKAADSTRPMTYGDNKIKANWNESNTMGDNLTANGGVVGTNYSDGANYDK
IRTTHPSWAIYGSETASAINSRGIYNRTTGGAQSSDKQLTSYDNSAVGWGAVASSAWYDVVQRDFVAGTYVWTGFDYLGEPTPWNGTGSGA
VGSWPSPKNSYFGIVDTAGFPKDTYYFYQSQWNDDVHTLHILPAWNENVVAKGSGNNVPVVVYTDAAKVKLYFTPKGSTEQRLIGEKSFTK
KTTAAGYTYQVYEGSDKDSTAHKNMYLTWNVPWAEGTISAEAYDENNRLIPEGSTEGNASVTTTGKAAKLKADADRKTITADGKDLSYIEV
DVTDANGHIVPDAANRVTFDVKGAGKLVGVDNGSSPDHDSYQADNRKAFSGKVLAIVQSTKEAGEITVTAKADGLQSSTVKIATTAVPGTS
TEKTVRSFYYSRNYYVKTGNKPILPSDVEVRYSDGTSDRQNVTWDAVSDDQIAKAGSFSVAGTVAGQKISVRVTMIDEIGALLNYSASTPV
GTPAVLPGSRPAVLPDGTVTSANFAVHWTKPADTVYNTAGTVKVPGTATVFGKEFKVTATIRVQRSQVTIGSSVSGNALRLTQNIPADKQS
DTLDAIKDGSTTVDANTGGGANPSAWTNWAYSKAGHNTAEITFEYATEQQLGQIVMYFFRDSNAVRFPDAGKTKIQISADGKNWTDLAATE
TIAAQESSDRVKPYTYDFAPVGATFVRVTVTNADTTTPSGVVCAGLTEIELKTATSKFVANTSAALSSLTVNGTKVSDSVLAAGSYNTPAI
IADVKAEGEGNASVTVLPAHDNVIRVITESEDHVTRKTFTINLGTEQEFPADSDERDQHQHQHQHQQ
SEQ ID NO: 10
MAVRRLGGRIVAFAATVALSIPLGLLTNSAWAVEDATRSDSTTQMSSTPEVVYSSAVDSKQNRTSDFDANWKFMLSDSVQAQDPAFDDSAW
QQVDLPHDYSITQKYSQSNEAESAYLPGGTGWYRKSFTIDRDLAGKRIAINFDGVYMNATVWFNGVKLGTHPYGYSPFSFDLTGNAKFGGE
NTIVVKVENRLPSSRWYSGSGIYRDVTLTVTDGVHVGNNGVAIKTPSLATQNGGDVTMNLTTKVANDTEAAANITLKQTVFPKGGKTDAAI
GTVTTASKSIAAGASADVTSTITAASPKLWSIKNPNLYTVRTEVLNGGKVLDTYDTEYGFRWTGFDATSGFSLNGEKVKLKGVSMHHDQGS
LGAVANRRAIERQVEILQKMGVNSIRTTHNPAAKALIDVCNEKGVLVVEEVFDMWNRSKNGNTEDYGKWFGQAIAGDNAVLGGDKDETWAK
FDLTSTINRDRNAPSVIMWSLGNEMMEGISGSVSGFPATSAKLVAWTKAADSTRPMTYGDNKIKANWNESNTMGDNLTANGGVVGTNYSDG
ANYDKIRTTHPSWAIYGSETASAINSRGIYNRTTGGAQSSDKQLTSYDNSAVGWGAVASSAWYDVVQRDFVAGTYVWTGFDYLGEPTPWNG
TGSGAVGSWPSPKNSYFGIVDTAGFPKDTYYFYQSQWNDDVHTLHILPAWNENVVAKGSGNNVPVVVYTDAAKVKLYFTPKGSTEKRLIGE
KSFTKKTTAAGYTYQVYEGSDKDSTAHKNMYLTWNVPWAEGTISAEAYDENNRLIPEGSTEGNASVTTTGKAAKLKADADRKTITADGKDL
SYIEVDVTDANGHIVPDAANRVTFDVKGAGKLVGVDNGSSPDHDSYQADNRKAFSGKVLAIVQSTKEAGEITVTAKADGLQSSTVKIATTA
VPGTSTEKTVRSFYYSRNYYVKTGNKPILPSDVEVRYSDGTSDRQNVTWDAVSDDQIAKAGSFSVAGTVAGQKISVRVTMIDEIGALLNYS
ASTPVGTPAVLPGSRPAVLPDGTVTSANFAVHWTKPADTVYNTAGTVKVPGTATVFGKEFKVTATIRVQRSQVTIGSSVSGNALRLTQNIP
ADKQSDTLDAIKDGSTTVDANTGGGANPSAWTNWAYSKAGHNTAEITFEYATEQQLGQIVMYFERDSNAVRFPDAGKTKIQISADGKNWTD
LAATETIAAQESSDRVKPYTYDFAPVGATFVKVTVTNADTTTPSGVVCAGLTEIELKTATSKFVTNTSAALSSLTVNGTKVSDSVLAAGSY
NTPAIIADVKAEGEGNASVTVLPAHDNVIRVITESEDHVTRKTFTINLGTEQEFPADSDERDYPAADMTVTVGSEQTSGTATEGPKKFAVD
GNTSTYWHSNWTPTTVNDLWIAFELQKPTKLDALRYLPRPAGSKNGSVTEYKVQVSDDGTNWTDAGSGTWTTDYGWKLAEFNQPVTTKHVR
LKAVHTYADSGNDKFMSASEIRLRKAVDTTDISGATVTVPAKLTVDRVDADHPATFATKDVTVTLGDATLRYGVDYLLDYAGNTAVGKATV
TVRGIDKYSGTVAKTFTIELKNAPAPEPTLTSVSVKTKPSKLTYVVGDAFDPAGLVLQHDRQADRPPQPLVGEQADERGLTCGTRCDRVEQ
LRKHENREAHRTGLDHLEFVGAADGAVGEQATFKVHVHADQGDGRHDDADERDIDPHVPVDHAVGELARAACHHVIGLRVDTHRLKASGFQ
IPADDMAEIDRITGFHRFERHVG
SEQ ID NO: 11
MAVRRLGGRIVAFAATVALSIPLGLLTNSAWAVEDATRSDSTTQMSSTPEVVYSSAVDSKQNRTSDFDANWKFMLSDSVQAQDPAFDDSAW
QQVDLPHDYSITQKYSQSNEAESAYLPGGTGWYRKSFTIDRDLAGKRIAINFDGVYMNATVWFNGVKLGTHPYGYSPFSFDLTGNAKEGGE
NTIVVKVENRLPSSRWYSGSGIYRDVTLTVTDGVHVGNNGVAIKTPSLATQNGGNVTMNLTTKVANDTKAAANITLKQTVFPKGGKTDAAI
GTVTTASKSIAAGASADVTSTITAASPKLWSIKNPNLYTVRTEVLNGGKVLDTYDTEYGFRWTGFDATSGFSLNGEKVKLKGVSMHHDQGS
LGAVANRRAIERQVEILQKMGVNSIRTTHNPAAKALIDVCNEKGVLVVEEVFDMWNRSKNGNTEDYGKWFGQAIAGDNAVLGGDKDETWAK
FDLTSTINRDRNAPSVIMWSLGNEMMEGISGSVSGFPATSAKLVAWTKAADSTRPMTYGDNKIKANWNESNTMGDNLTANGGVVGTNYSDG
ANYDKIRTTHPSWAIYGSETASAINSRGIYNRTTGGAQSSDKQLTSYDNSAVGWGAVASSAWYDVVQRDFVAGTYVWTGFDYLGEPTPWNG
TGSGAVGSWPSPKNSYFGIVDTAGFPKDTYYFYQSQWNDDVHTLHILPAWNENVVAKGSGNNVPVVVYTDAAKVKLYFTPKGSTEKRLIGE
KSFTKKTTAAGYTYQVYEGADKDSTAHKNMYLTWNVPWAEGTISAEAYDENNRLIPEGSTEGNASVTTTGKAAKLKADADRKTITADGKDL
SYIEVDVTDANGHIVPDAANRVTFDVKGAGKLVGVDNGSSPDHDSYQADNRKAFSGKVLAIVQSTKEAGEITVTAKADGLQSSTVKIATTA
VPGTSTEKTVRSFYYSRNYYVKTGNKPILPSDVEVRYSDGTSDRQNVTWDAVSDDQIAKAGSFSVAGTVAGQKISVRVTMIDEIGALLNYS
ASTPVGTPAVLPGSRPAVLPDGTVTSANFAVDWTKPADTVYNTAGTVKVPGTATVFGKEFKVTATIRVQRSQVTIGSSVSGNALRLTQNIP
ADKQSDTLDAIKDGSTTVDANTGGGANPSAWTNWAYSKAGHNTAEITFEYATEQQLGQIVMYFFRDSNAVRFPDAGKTKIQISADGKNWTD
LAATETIAAQESSDRVKPYTYDFAPVGATFVKVTVTNADTTTPSGVVCAGLTEIELKTATSKFVTNTSAALSSLTVNGTKVSDSVLAAGSY
NTPAIIADVKAEGEGNASVTVLPAHDNVIRVITESEDHVTRKTFTINLGTEQEFPADSDERDYPAADMTVTAGSEQTSGTATEGPKKFAVD
GNTSTYWHSNWTPTTVNDLWIAFELQKPTKLDALRYLPRPAGSKNGSVTEYKVQVSDDGTNWTDAGSGTWTTDYGWKLAEFNQPVTTKHVR
LKAVHTYADSGNDKFMSASEIRLRKAVDTTDISGATVTVPAKLTVDRVDADHPATFATKDVTVTLGDATLRYGVDYLLDYAGNTAVGKATV
TVRGIDKYSGTVAKTFTIELKNAPAPEPTLTSVSVKTKPSKLTYVVGDAFDPAGLVLQLNYDDDSTGTVTWNTQTAGDFTFKPALDAKLKV
TDKTVTVTYQGKSAVIDITVSQPAPTVSKTDLDKAIKAIEAKNPDSSKYTADSWKTFADAMAHAKAVIADDSATQQDVDKALKALTDAYAG
LTEKTPEPAPVSKSELDKKIKAIEAEKLDGSKYTAESWKAFETALAHAKAVIASDSATQQDVDAALGALTSARDGLTEKGEVKPDPKPEPG
TVDKAALDKAVKKVEAEKLDGSKYTADSWKAFETALAHAKAVIGNANSTQFDIDNALSMLNDARAALKEKPGRIIAIIDGGALSKTGASVA
IIASVAAAMKAVGAGVMALRPPKW
SEQ ID NO: 12
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKT
SEQ ID NO: 13
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGAL
SEQ ID NO: 14
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQ
SEQ ID NO: 15
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQI
SEQ ID NO: 16
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKEDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSDRVKPYTYDFAPVGATFVK
VTVTNADTTTPSGVVCAGLTEIELKTAT
SEQ ID NO: 17
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSDRVKPYTYDFAPVGATFVK
VTVTNADTTTPSGVVCAGLTEIELKTATSKFVTNTSAALSSLTVNGTKVSDSVLAAGSYNTPAIIADVKAEGEGNASVTVLPAHDNVIRVI
TESEDHVTRKTFTINLGTEQEF
SEQ ID NO: 18
VEDATRSDSTTQMSSTPEVVYSSAVDSKQNRTSDFDANWKFMLSDSVQAQDPAFDDSAWQQVDLPHDYSITQKYSQSNEAESAYLPGGTGW
YRKSFTIDRDLAGKRIAINFDGVYMNATVWFNGVKLGTHPYGYSPFSFDLTGNAKFGGENTIVVKVENRLPSSRWYSGSGIYRDVTLTVTD
GVHVGNNGVAIKTPSLATQNGGDVTMNLTTKVANDTEAAANITLKQTVFPKGGKTDAAIGTVTTASKSIAAGASADVTSTITAASPKLWSI
KNPNLYTVRTEVLNGGKVLDTYDTEYGFRWTGFDATSGFSLNGEKVKLKGVSMHHDQGSLGAVANRRAIERQVEILQKMGVNSIRTTHNPA
AKALIDVCNEKGVLVVEEVFDMWNRSKNGNTEDYGKWFGQAIAGDNAVLGGDKDETWAKFDLTSTINRDRNAPSVIMWSLGNEMMEGISGS
VSGFPATSAKLVAWTKAADSTRPMTYGDNKIKANWNESNTMGDNLTANGGVVGTNYSDGANYDKIRTTHPSWAIYGSETASAINSRGIYNR
TTGGAQSSDKQLTSYDNSAVGWGAVASSAWYDVVQRDFVAGTYVWTGFDYLGEPTPWNGTGSGAVGSWPSPKNSYFGIVDTAGFPKDTYYF
YQSQWNDDVHTLHILPAWNENVVAKGSGNNVPVVVYTDAAKVKLYFTPKGSTEKRLIGEKSFTKKTTAAGYTYQVYEGSDKDSTAHKNMYL
TWNVPWAEGTISAEAYDENNRLIPEGSTEGNASVTTTGKAAKLKADADRKTITADGKDLSYIEVDVTDANGHIVPDAANRVTFDVKGAGKL
VGVDNGSSPDHDSYQADNRKAFSGKVLAIVQSTKEAGEITVTAKADGLQSSTVKIATTAVPGTSTEKTVRSFYYSRNYYVKTGNKPILPSD
VEVRYSDGTSDRQNVTWDAVSDDQIAKAGSFSVAGTVAGQKISVRVTMIDEIGALLNYSASTPVGTPAVLPGSRPAVLPDGTVTSANFAVH
WTKPADTVYNTAGTVKVPGTATVFGKEFKVTATIRVQRSQVTIGSSVSGNALRLTQNIPADKQSDTLDAIKDGSTTVDANTGGGANPSAWT
NWAYSKAGHNTAEITFEYATEQQLGQIVMYFFRDSNAVRFPDAGKTKIQISADGKNWTDLAATETIAAQESSDRVKPYTYDFAPVGATFVK
VTVTNADTTTPSGVVCAGLTEIELKTATSKFVTNTSAALSSLTVNGTKVSDSVLAAGSYNTPAIIADVKAEGEGNASVTVLPAHDNVIRVI
TESEDHVTRKTFTINLGTEQEFPADSDERDYPAADMTVTVGSEQTSGTATEGPKKFAVDGNTSTYWHSNWTPTTVNDLWIAFELQKPTKLD
ALRYLPRPAGSKNGSVTEYKVQVSDDGTNWTDAGSGTWTTDYGWKLAEFNQPVTTKHVRLKAVHTYADSGNDKFMSASEIRLRKAVDTTDI
SGATVTVPAKLTVDRVDADHPATFATKDVTVTLGDATLRYGVDYLLDYAGNTAVGKATVTVRGIDKYSGTVAKTFTIELKNAPAPEPTLTS
VSVKTKPSKLTYVVGDAFDPAGLVLQHDRQADRPPQPLVGEQADERGLTCGTRCDRVEQLRKHENREAHRTGLDHLEFVGAADGAVGEQAT
FKVHVHADQGDGRHDDADERDIDPHVPVDHAVGELARAACHHVIGLRVDTHRLKASGFQIPADDMAEIDRITGFHRFERHVG

### REFERENCES

WO2010149557, WO2016071500, WO2021180702, WO2013182686, WO2015132402, WO2020117548, WO2018210820

1. A spray dried milk powder composition or spray dried milk-based powder composition comprising:
a beta-galactosidase enzyme having transgalactosylating activity and at least 90% sequence identity to SEQ ID NOs 1, 2, 3, 4, 5, 6, 7, 8, 9, 10, 11, 12, 13, 14, 15, 16, 17 or 18;
galacto-oligosaccharides;
less than 5% w/w of lactose;
wherein the composition is substantially free of maltodextrin; and
substantially free of sodium chloride.

2. The composition of claim 1, comprising at least 8% w/w of galacto-oligosaccharides, wherein w/w is w_{galacto-oligosaccharides}/w_{composition}.

3. The composition of claim 1, comprising 8%-30% w/w of galacto-oligosaccharides, wherein w/w is w_{galacto-oligosaccharides}/w_{composition}.

4. The composition of claim 1, comprising 4.5% w/w or less of lactose, wherein w/w is w_{lactose}/w_{composition}.

5. The composition of claim 1, wherein the beta-galactosidase enzyme has at least 95% sequence identity to SEQ ID NOs 1, 2, 3, 4, 5, 6, 7, 8, 9, 10, 11, 12, 13, 14, 15, 16, 17 or 18.

6. A method for producing a spray-dried milk powder composition or spray dried milk-based powder composition according to claim 1, wherein the method comprises the following steps:
providing a milk or milk-based substrate comprising at least 1% w_{lactose}/w_{milk or milk-based substrate} and at most 1.5% w_{fat}/w_{milk or milk-based substrate};
mixing the milk or milk-based substrate with a beta-galactosidase enzyme having transgalactosylating activity and at least 90% sequence identity to SEQ ID NOs 1, 2, 3, 4, 5, 6, 7, 8, 9, 10, 11, 12, 13, 14, 15, 16, 17 or 18;
allowing the beta-galactosidase enzyme to generate galacto-oligosaccharides;
spray drying the mix comprising milk or milk-based substrate, beta-galactosidase enzyme and galacto-oligosaccharides;
obtaining a spray dried milk powder composition or milk-based substrate powder composition comprising
the beta-galactosidase enzyme,
galacto-oligosaccharides,
less than 5% w_{lactose}/w_{composition},
wherein the composition is substantially free of maltodextrin; and
wherein the composition is substantially free of sodium chloride.

7. The method according to claim 6, wherein the composition comprises at least 8% w/w of galacto-oligosaccharides, wherein w/w is w_{galacto-oligosaccharides}/w_{composition}.

8. The method according to claim 6, wherein the composition comprises 8%-30% w/w of galacto-oligosaccharides, wherein w/w is w_{galacto-oligosaccharides}/w_{composition}.

9. The method according to claim 6, wherein the composition comprises 4.5% w/w or less of lactose, wherein w/w is w_{lactose}/w_{composition}.

10. The method according to claim 6, wherein the step of mixing the milk or milk-based substrate with the beta-galactosidase enzyme is carried out with at least 0.1 g/L, wherein g/L is g_{beta-galactosidase of enzyme}/Lₘᵢₗₖ or g_{beta-galactosidase of enzyme}/L_{milk-substrate}.

11. The method according to claim 6, wherein the beta-galactosidase enzyme has at least 95% sequence identity to SEQ ID NOs 1, 2, 3, 4, 5, 6, 7, 8, 9, 10, 11, 12, 13, 14, 15, 16, 17 or 18.

12. The method according to claim 6, wherein the method is carried out in the absence of a spray drying aid.

13. The method according to claim 6, wherein the step of allowing the beta-galactosidase enzyme to generate galacto-oligosaccharides is carried out between 1 h-24 h.

14. The method according to claim 6, wherein the milk or milk-based substrate has 1.5%-0.1% w_{fat}/w_{milk or milk-based substrate}.

15. Use of a beta-galactosidase enzyme having transgalactosylating activity for improving spray drying of milk or milk-based substrate or for improving mass yield of a spray dried milk powder composition or spray dried milk-based powder composition or for reducing browning of a spray dried milk or milk-based powder composition.

16. The use according to claim 15, wherein the beta-galactosidase enzyme is a Bifidobacterium beta-galactosidase.

17. A method for producing a spray-dried milk powder composition or spray dried milk-based powder composition, wherein the method comprises the following steps:
a) providing an aqueous composition comprising less than 30% by weight of non-fat milk solids including lactose;
b) adding a beta-galactosidase to the composition;
c) allowing the beta-galactosidase to react for 10-240 minutes at 50-65° C.;
d) treating the composition after step c) at a temperature of below 70° C. for 10-150 seconds;
e) spray drying the composition after step d).

18. The composition of claim 1, wherein the galacto-oligosaccharides are at least 1% w_{galacto-oligosaccharides}/w_{composition}.

19. The composition of claim 2, comprising at least 10% w/w of galacto-oligosaccharides.

20. The composition of claim 3, comprising 11%-20% w/w of galacto-oligosaccharides.


SEQUENCE LISTING (18 sequences)

SEQ ID NO: 1 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 2 - protein, 965 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 3 - protein, 1038 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 4 - protein, 1142 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 5 - protein, 1211 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 6 - protein, 1296 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEF

SEQ ID NO: 7 - protein, 1304 aa, synthetic construct
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 8 - protein, 1931 aa, synthetic construct
MKKPLGKIVASTALLISVAFSSSIASAAVEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDNALKALTDAYAGLTEKTPEPAPVSKSELDKKIKAIEAEKLDGSKYTAESWKAFETALAHAKAVIASDSATQQNVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGSALSKTGASVAIIASVAAAMLAVGAGVMALRRKRS

SEQ ID NO: 9 - protein, 1341 aa, synthetic construct
MKKPLGKIVASTALLISVAFSSSIASAIEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFSATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEQRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVRVTVTNADTTTPSGVVCAGLTEIELKTATSKFVANTSAALSSLTVNGTKVSDSVLAAGSYNTPAIIADVKAEGEGNASVTVLPAHDNVIRVITESEDHVTRKTFTINLGTEQEFPADSDERDQHQHQHQHQQ

SEQ ID NO: 10 - protein, 1752 aa, synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 11 - protein, 1935 aa, synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTAGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDKALKALTDAYAGLTEKTPEPAPVSKSELDKKIKAIEAEKLDGSKYTAESWKAFETALAHAKAVIASDSATQQDVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGGALSKTGASVAIIASVAAAMKAVGAGVMALRPPKW

SEQ ID NO: 12 - protein, 887 aa, Bifidobacterium bifidum; identical to SEQ ID NO: 1

SEQ ID NO: 13 - protein, 965 aa, Bifidobacterium bifidum; identical to SEQ ID NO: 2

SEQ ID NO: 14 - protein, 1038 aa, Bifidobacterium bifidum; identical to SEQ ID NO: 3

SEQ ID NO: 15 - protein, 1142 aa, Bifidobacterium bifidum; identical to SEQ ID NO: 4

SEQ ID NO: 16 - protein, 1211 aa, Bifidobacterium bifidum; identical to SEQ ID NO: 5

SEQ ID NO: 17 - protein, 1296 aa, Bifidobacterium bifidum; identical to SEQ ID NO: 6

SEQ ID NO: 18 - protein, 1720 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG